(19) United States
(12) Patent Application Publication
(10) Pub. No.: US 2019/0262291 A1
(43) Pub. Date: Aug. 29, 2019

(54) METHODS AND COMPOSITIONS FOR ALLEVIATING MYOPATHY

(71) Applicant: Roland W. WINTERFIELD, Hinsdale, IL (US)

(72) Inventor: Roland W. WINTERFIELD, Hinsdale, IL (US)

(21) Appl. No.: 16/318,023

(22) PCT Filed: Jul. 19, 2017
(86) PCT No.: PCT/US2017/042881

§ 371 Date: Jan. 15, 2019

Related U.S. Application Data
(60) Provisional application No. 62/364,100, filed on Jul. 19, 2016.

(51) Int. Cl. A61K 31/19; A61K 31/366

(52) U.S. Cl. CPC A61K 31/19; A61K 31/366

(57) ABSTRACT

Disclosed herein are methods and compositions for alleviating side effects of statin administration, such as myopathic or myalgic side effects, short-term memory loss, abnormal liver function, glucose intolerance, hyperglycemia, increased risk for diabetes, or cumulative trauma disorder, comprising administration of β-hydroxy β-methylbutyrate (HMB) and leucic acid to an individual taking a statin. Also disclosed are methods and compositions for alleviating acute rhabdomyolysis, and methods and compositions for treating cumulative trauma disorder, comprising administration of HMB and leucic acid. The disclosure further provides uses of HMB and leucic acid in combination with a statin to alleviate side effects of statin administration, treat statin intolerance, treat cumulative trauma disorder, and treat acute rhabdomyolysis.

### BACKGROUND

HMG-CoA reductase inhibitors, commonly known as statins, are a class of drugs used to lower cholesterol levels by inhibiting the enzyme HMG-CoA reductase, which catalyzes the rate-limiting conversion of HMG-CoA into mevalonate by HMG-CoA reductase during de novo cholesterol biosynthesis. Statins are used primarily to treat hyperlipidemias and are the most effective lipid-lowering drugs currently available. They have also been shown to exhibit pleiotropic effects and may have potential uses in the treatment of other conditions, such as diabetes, depression, cancer, osteoporosis, ventricular arrhythmias, peripheral arterial disease, and idiopathic dilated cardiomyopathy.

Side effects of statins include myopathy (including myalgia), increased risk of diabetes, short-term memory loss, cumulative trauma disorder (also known as chronic overuse syndrome or repetitive overuse syndrome), and statin-induced hepatic trans-aminitis (evidenced by abnormalities in liver enzyme tests). Myopathy is the most common side effect, with symptoms that can include muscle fatigue, weakness, pain, and rhabdomyolysis (i.e., the breakdown of muscle fibers that leads to the release of muscle fiber contents (inter alia, myoglobin) into the bloodstream). Rhabdomyolysis is rare, occurring in ˜0.1% of patients; the occurrence of other myopathic symptoms has been estimated at 1-5% of patients in controlled studies using selected patients with 35% of eligible patients excluded (LaRosa et al., New England Journal of Medicine 2005, 352: 1425-35). An observational study (PRIMO) involving 7924 French unselected outpatients on statin therapy, reported 10.5% of statin users experienced statin-related myalgia/myopathy (Bruckert et al., 2005, Cardiovascular Drugs and Therapy 19: 403-14). Other observational studies have estimated that 9-20% of statin users experience statin-related muscle symptoms. Physical exercise appears to exacerbate the incidence of myalgia, with as many as 25% of statin users who exercise experiencing muscle fatigue, weakness, aches, and cramping.

Thus, there is a need in the art to improve treatment of statin-related diseases and disorders by alleviating said deleterious side effects.

### SUMMARY

This disclosure provides certain advantages and advancements over the prior art, in particular, methods for alleviating statin-induced myopathy and/or myalgia (SIM) comprising administering β-hydroxy β-methylbutyrate (HMB) in combination with leucic acid to an individual taking a statin. In alternative embodiments, the disclosure provides methods for alleviating myopathy and/or myalgia, for treating acute rhabdomyolysis, and for treating cumulative trauma disorder in individuals not taking a statin comprising administering HMB in combination with leucic acid.

In one aspect, the disclosure provides methods for alleviating one or more side effects of statin administration, the methods comprising supplementing statin administration with administration of a therapeutically effective amount of β-hydroxy β-methylbutyrate (HMB), in combination with a therapeutically effective amount of leucic acid. In another aspect, the disclosure provides methods for alleviating one or more side effects of statin administration comprising co-administering β-hydroxy β-methylbutyrate (HMB) and leucic acid. In some embodiments, the one or more side effects of statin administration are myopathic or myalgic side effects, short-term memory loss, elevated alanine transaminase (ALT) or aspartate transaminase (AST) levels, glucose intolerance, hyperglycemia, increased risk for diabetes, or cumulative trauma disorder.

In some embodiments of this aspect, HMB is administered at a dosage of approximately 2.0 to 4.0 grams/day, and/or leucic acid is administered at a dosage of approximately 1.0 to 4.0 grams/day. In some embodiments, HMB is administered at a dosage of approximately 3.0 grams/day. In some embodiments, HMB is administered at a dosage of approximately 4.0 grams/day. In some embodiments, leucic acid is administered at a dosage of approximately 1.5 grams/day. In some embodiments, leucic acid is administered at a dosage of approximately 3.0 grams/day. In some embodiments, HMB is administered 1 to 5 times per day, and/or leucic acid is administered 1 to 5 times per day. In some embodiments, HMB is administered 3 times per day. In some embodiments, HMB is administered 2 times per day. In some embodiments, leucic acid is administered 2 times per day. In some embodiments, leucic acid is administered once per day.

In some embodiments of this aspect, the statin is atorvastatin, cerivastatin, fluvastatin, lovastatin, mevastatin, pitavastatin, pravastatin, rosuvastatin, or simvastatin. In some embodiments, the statin is administered at a dosage of: (a) 10 to 80 mg atorvastatin; (b) 5 to 40 mg rosuvastatin; (c) 10 to 80 mg pravastatin; (d) 5 to 80 mg simvastatin; (e) 10 to 80 mg lovastatin; (f) 1 to 4 mg pitavastatin; or (g) 20 to 80 mg fluvastatin. In some embodiments, the statin is administered at a dosage of: (a) 10 mg, 20 mg, or 40 mg atorvastatin; (b) 5 mg, 10 mg, 20 mg, or 40 mg rosuvastatin; (c) 10 mg, 20 mg, 40 mg, or 80 mg pravastatin; (d) 10 mg, 20 mg, or 40 mg simvastatin; (e) 10 mg, 20 mg, 40 mg, or 80 mg lovastatin; (f) 1 mg, 2 mg, or 4 mg pitavastatin; or (g) 20 mg, 40 mg, or 80 mg fluvastatin.

In some embodiments of this aspect, HMB is administered as calcium HMB monohydrate. In some embodiments, HMB is administered as HMB free acid. In some embodiments, leucic acid is administered as leucic acid sodium salt. In some embodiments, HMB and leucic acid are administered as a conjugate.

In another aspect, the disclosure provides methods for treating statin intolerance, the methods comprising supplementing statin administration with administration of a therapeutically effective amount of β-hydroxy β-methylbutyrate (HMB), in combination with a therapeutically effective amount of leucic acid. In another aspect, the disclosure provides methods for treating statin intolerance, the methods comprising co-administering therapeutically effective amounts of β-hydroxy β-methylbutyrate (HMB) and leucic acid. In some embodiments, statin intolerance comprises muscle aches, pains, weakness, or cramps. In some embodiments, HMB is administered at a dosage of approximately 2.0 to 4.0 grams/day, and wherein leucic acid is administered at a dosage of approximately 1.0 to 4.0 grams/day. In some embodiments, HMB is administered at a dosage of approximately 3.0 grams/day. In some embodiments, HMB is administered at a dosage of approximately 4.0 grams/day. In some embodiments, leucic acid is administered at a dosage of approximately 1.5 grams/day. In some embodiments, leucic acid is administered at a dosage of approximately 3.0 grams/day. In some embodiments, HMB is administered 1 to 5 times per day, and wherein leucic acid is administered 1 to 5 times per day. In some embodiments, HMB is administered 3 times per day. In some embodiments, HMB is administered 2 times per day. In some embodiments, leucic acid is administered 2 times per day. In some embodiments, leucic acid is administered once per day.

In some embodiments of this aspect, the statin is atorvastatin, cerivastatin, fluvastatin, lovastatin, mevastatin, pitavastatin, pravastatin, rosuvastatin, or simvastatin. In some embodiments, the statin is administered at a dosage of: (a) 10 to 80 mg atorvastatin; (b) 5 to 40 mg rosuvastatin; (c) 10 to 80 mg pravastatin; (d) 5 to 80 mg simvastatin; (e) 10 to 80 mg lovastatin; (f) 1 to 4 mg pitavastatin; or (g) 20 to 80 mg fluvastatin. In some embodiments, the statin is administered at a dosage of: (a) 10 mg, 20 mg, or 40 mg atorvastatin; (b) 5 mg, 10 mg, 20 mg, or 40 mg rosuvastatin; (c) 10 mg, 20 mg, 40 mg, or 80 mg pravastatin; (d) 10 mg, 20 mg, or 40 mg simvastatin; (e) 10 mg, 20 mg, 40 mg, or 80 mg lovastatin; (f) 1 mg, 2 mg, or 4 mg pitavastatin; or (g) 20 mg, 40 mg, or 80 mg fluvastatin.

In some embodiments of this aspect, HMB is administered as calcium HMB monohydrate. In some embodiments, HMB is administered as HMB free acid. In some embodiments, leucic acid is administered as leucic acid sodium salt. In some embodiments, HMB and leucic acid are administered as a conjugate.

In another aspect, the disclosure provides pharmaceutical formulations comprising therapeutically effective amounts of a statin, β-hydroxy β-methylbutyrate (HMB), and leucic acid, wherein side effects or intolerance induced by administration of only the statin are reduced or alleviated. In some embodiments, the amounts of HMB and leucic acid are sufficient to alleviate one or more side effects of the statin. In some embodiments, the statin is atorvastatin, cerivastatin, fluvastatin, lovastatin, mevastatin, pitavastatin, pravastatin, rosuvastatin, or simvastatin. In some embodiments, the pharmaceutical formulations comprise: (a) 10 to 80 mg atorvastatin; (b) 5 to 40 mg rosuvastatin; (c) 10 to 80 mg pravastatin; (d) 5 to 80 mg simvastatin; (e) 10 to 80 mg lovastatin; (f) 1 to 4 mg pitavastatin; or (g) 20 to 80 mg fluvastatin. In some embodiments, the pharmaceutical formulations comprise: (a) 10 mg, 20 mg, or 40 mg atorvastatin; (b) 5 mg, 10 mg, 20 mg, or 40 mg rosuvastatin; (c) 10 mg, 20 mg, 40 mg, or 80 mg pravastatin; (d) 10 mg, 20 mg, or 40 mg simvastatin; (e) 10 mg, 20 mg, 40 mg, or 80 mg lovastatin; (f) 1 mg, 2 mg, or 4 mg pitavastatin; or (g) 20 mg, 40 mg, or 80 mg fluvastatin.

In some embodiments of this aspect, the formulations comprise statin and HMB at a statin-to-HMB ratio that is approximately 0.001 to 0.1 by weight, and the formulations comprise statin and leucic acid at a statin-to-leucic acid ratio that is approximately 0.001 to 0.1 by weight. In some embodiments, the formulations comprise from about 1.0 gram to about 4.0 grams HMB. In some embodiments, the formulations comprise from about 1.0 gram to about 4.0 grams leucic acid. In some embodiments, HMB is calcium HMB monohydrate. In some embodiments, HMB is HMB free acid. In some embodiments, leucic acid is leucic acid sodium salt. In some embodiments, HMB is conjugated to leucic acid.

In another aspect, the disclosure provides methods for treating acute rhabdomyolysis in a patient, the methods comprising administering a therapeutically effective amount of β-hydroxy β-methylbutyrate (HMB) and a therapeutically effective amount of leucic acid to the patient. In some embodiments, HMB is administered at a dosage of about 6 grams/day to about 12 grams/day, and leucic acid is administered at a dosage of about 1 gram/day to about 3 grams/day. In some embodiments, HMB is administered at a dosage of about 12 grams/day. In some embodiments, leucic acid is administered at a dosage of about 1.5 grams/day. In some embodiments, HMB is HMB free acid. In some embodiments, leucic acid is leucic acid sodium salt. In some embodiments, HMB and leucic acid are administered for at least three days. In some embodiments, HMB and leucic acid are administered as a conjugate.

In another aspect, the disclosure provides methods for treating cumulative trauma disorder in a patient, the method comprising administering a therapeutically effective amount of β-hydroxy β-methylbutyrate (HMB) and a therapeutically effective amount of leucic acid to the patient. In some embodiments, the cumulative trauma disorder is associated with statin use. In some embodiments, HMB is administered at a dosage of approximately 2.0 to 4.0 grams/day, and wherein leucic acid is administered at a dosage of approximately 1.0 to 4.0 grams/day. In some embodiments, HMB is administered at a dosage of approximately 3.0 grams/day. In some embodiments, HMB is administered at a dosage of approximately 4.0 grams/day. In some embodiments, leucic acid is administered at a dosage of approximately 1.5 grams/day. In some embodiments, leucic acid is administered at a dosage of approximately 3.0 grams/day. In some embodiments, HMB is administered 1 to 5 times per day, and wherein leucic acid is administered 1 to 5 times per day. In some embodiments, HMB is administered 3 times per day. In some embodiments, HMB is administered 2 times per day. In some embodiments, leucic acid is administered 2 times per day. In some embodiments, leucic acid is administered once per day. In some embodiments, HMB is HMB free acid or calcium HMB monohydrate. In some embodiments, leucic acid is leucic acid sodium salt. In some embodiments, HMB and leucic acid are administered for at least three weeks. In some embodiments, HMB and leucic acid are administered for at least six weeks. In some embodiments, HMB and leucic acid are administered as a conjugate.

In another aspect, the disclosure provides uses of β-hydroxy β-methylbutyrate (HMB) in combination with leucic acid to alleviate one or more side effects in a patient administered a statin. In some embodiments, the one or more side effects of statin administration are myopathic or myalgic side effects, short-term memory loss, elevated alanine transaminase (ALT) or aspartate transaminase (AST) levels, glucose intolerance, hyperglycemia, increased risk for diabetes, or cumulative trauma disorder. In another aspect, the disclosure provides uses of β-hydroxy β-methylbutyrate (HMB) in combination with leucic acid to treat statin intolerance. In some embodiments, statin intolerance comprises muscle aches, pains, weakness, or cramps. In some embodiments, HMB is administered at a dosage of approximately 2.0 grams/day to approximately 4.0 grams/day, and leucic acid is administered at a dosage of approximately 1.0 grams/day to approximately 3.0 grams/day. In some embodiments, HMB is administered from 1 to 4 times per day, and wherein leucic acid is administered from 1 to 4 times per day. In some embodiments, the statin is atorvastatin, cerivastatin, fluvastatin, lovastatin, mevastatin, pitavastatin, pravastatin, rosuvastatin, or simvastatin. In some embodiments, the statin is administered at a dosage of approximately: (a) 10 to 80 mg atorvastatin; (b) 5 to 40 mg rosuvastatin; (c) 10 to 80 mg pravastatin; (d) 5 to 80 mg simvastatin; (e) 10 to 80 mg lovastatin; (f) 1 to 4 mg pitavastatin; or (g) 20 to 80 mg fluvastatin. In some embodiments, the statin is administered at a dosage of approximately: (a) 10 mg, 20 mg, or 40 mg atorvastatin; (b) 5 mg, 10 mg, 20 mg, or 40 mg rosuvastatin; (c) 10 mg, 20 mg, 40 mg, or 80 mg pravastatin; (d) 10 mg, 20 mg, or 40 mg simvastatin; (e) 10 mg, 20 mg, 40 mg, or 80 mg lovastatin; (f) 1 mg, 2 mg, or 4 mg pitavastatin; or (g) 20 mg, 40 mg, or 80 mg fluvastatin. In some embodiments, HMB is calcium HMB monohydrate. In some embodiments, HMB is HMB free acid. In some embodiments, leucic acid is leucic acid sodium salt. In some embodiments, HMB is conjugated to leucic acid.

In another aspect, the disclosure provides uses of β-hydroxy β-methylbutyrate (HMB) in combination with leucic acid to treat cumulative trauma disorder which may be, but which is not necessarily associated with statin administration. In some embodiments, HMB is administered at a dosage of approximately 2.0 grams/day to approximately 4.0 grams/day, and leucic acid is administered at a dosage of approximately 1.0 grams/day to approximately 3.0 grams/day. In some embodiments, HMB is administered from 1 to 4 times per day, and wherein leucic acid is administered from 1 to 4 times per day. In some embodiments, HMB is calcium HMB monohydrate. In some embodiments, HMB is HMB free acid. In some embodiments, leucic acid is leucic acid sodium salt. In some embodiments, HMB is conjugated to leucic acid.

In another aspect, the disclosure provides uses of β-hydroxy β-methylbutyrate (HMB) in combination with leucic acid to treat acute rhabdomyolysis. In some embodiments, HMB is administered at a dosage of approximately 6.0 grams/day to approximately 12.0 grams/day, and leucic acid is administered at a dosage of approximately 1.0 grams/day to approximately 3.0 grams/day. In some embodiments, HMB is administered from 1 to 4 times per day, and wherein leucic acid is administered from 1 to 4 times per day. In some embodiments, HMB is calcium HMB monohydrate. In some embodiments, HMB is HMB free acid. In some embodiments, leucic acid is leucic acid sodium salt. In some embodiments, HMB is conjugated to leucic acid.

These and other features and advantages of the present invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, in which:

FIG. 1 is a schematic of the human cholesterol biosynthesis pathway. Statins inhibit the initial step (conversion of HMG-CoA to mevalonic acid by HMG-CoA reductase), thereby preventing the downstream metabolic cascade. HMB reverses this inhibition, allowing isoprenoid production, the ubiquinone pathway and on-site myocyte cholesterol synthesis to proceed.

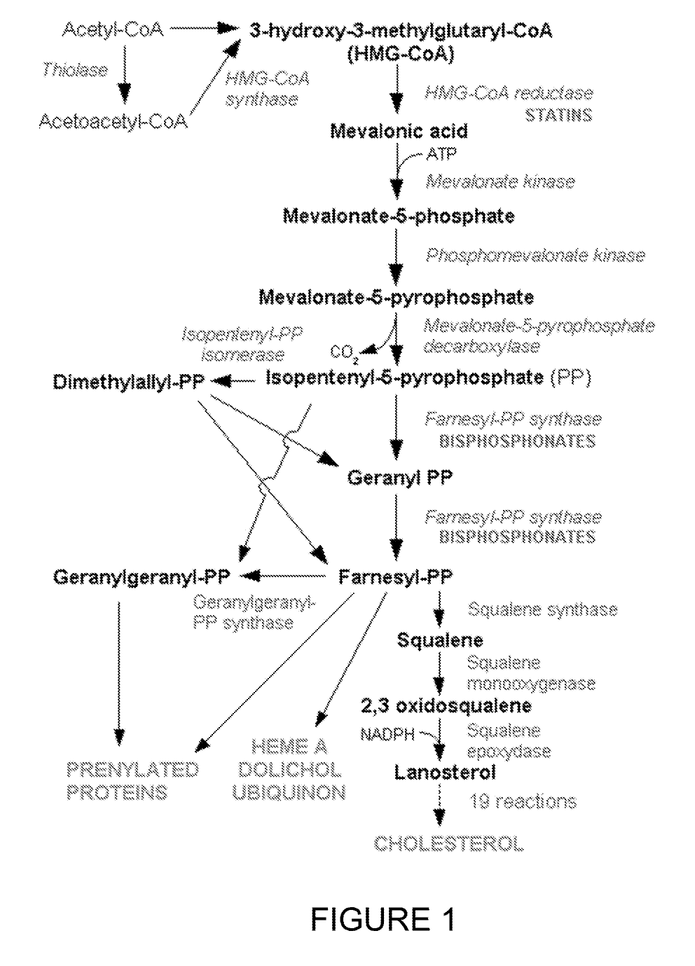

FIG. 2 shows the structure of a mammalian cell membrane. Cholesterol is an integral cell membrane component and is synthesized in situ to maintain cellular structural integrity, particularly in myocytes. In situ cholesterol biosynthesis is a process inhibited by statins but promoted by HMB.

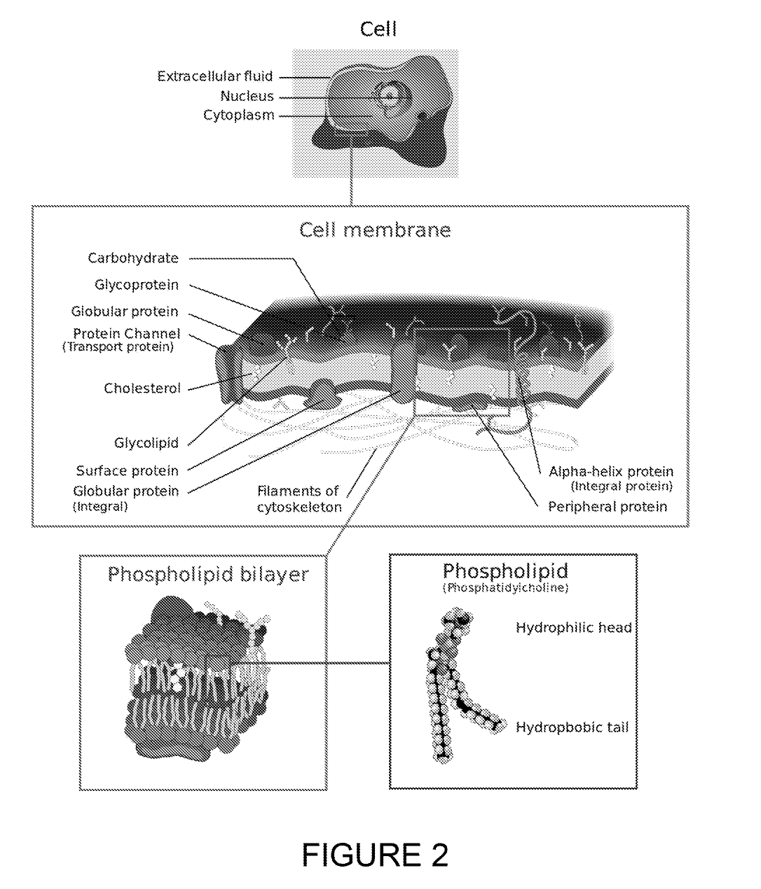

FIG. 3A shows the chemical structure of cholesterol. FIG. 3B shows a molecular stick model of cholesterol.

FIG. 4 depicts a molecule of cholesterol between two phospholipid molecules within a lipid bilayer.

FIG. 5 is a schematic of some elements of leucine, α-ketoisocaproate (KIC), and HMB metabolism in mammals, which comprises an alternative pathway in the myocyte for production of HMG CoA. HMB is converted to HMB-CoA, then to β-hydroxy-β-methylglutaryl-CoA (HMG-CoA), the conversion of which into mevalonate is catalyzed by HMG-CoA reductase. In a parallel pathway, leucine is metabolized to leucic acid, which is eventually converted to HMG-CoA. Mevalonate is eventually converted into cholesterol, as shown in FIG. 1. MC-CoA refers to β-methyl-crotonyl-CoA; MG-CoA refers to β-methyl-gluconyl-CoA.

Skilled artisans will appreciate that elements in the Figures are illustrated for simplicity and clarity and have not necessarily been drawn to scale. For example, the dimensions of some of the elements in the Figures can be exaggerated relative to other elements to help improve understanding of the embodiment(s) of the present invention.

### DETAILED DESCRIPTION

All publications, patents and patent applications cited herein are hereby expressly incorporated by reference for all purposes.

Before describing the present invention in detail, a number of terms will be defined. As used herein, the singular forms “a”, “an”, and “the” include plural referents unless the context clearly dictates otherwise. For example, reference to a “protein” means one or more proteins.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the term “statin” refers to a 3-hydroxy-3-methylglutaryl coenzyme A (HMG-CoA) reductase inhibitor. Statins block the rate-limiting step in de novo cholesterol biosynthesis, namely, the conversion of HMG-CoA into mevalonate by HMG-CoA reductase. Statins are used primarily as cholesterol-lowering (specifically, low-density lipoprotein (LDL)-lowering) medications to treat hyperlipidemias, such as hypercholesterolemia. Examples of statins with brand names and typical daily adult dose ranges provided in parentheses include: atorvastatin (LIPITOR®) (10-80 mg), fluvastatin (LESCOL®) (20-80 mg), lovastatin (MEVACOR®) (10-80 mg), pitavastatin (LIVALO®) (1-4 mg), pravastatin (PRAVACHOL®) (10-80 mg), rosuvastatin (CRESTOR®) (5-40 mg), and simvastatin (ZOCOR®) (5-80 mg). In some embodiments, the compositions and methods disclosed herein comprise statin doses of: 10 mg, 20 mg, or 40 mg atorvastatin or a pharmaceutically acceptable salt thereof; 5 mg, 10 mg, 20 mg, or 40 mg rosuvastatin or a pharmaceutically acceptable salt thereof; 10 mg, 20 mg, 40 mg, or 80 mg pravastatin or a pharmaceutically acceptable salt thereof; 10 mg, 20 mg, or 40 mg simvastatin or a pharmaceutically acceptable salt thereof; 10 mg, 20 mg, 40 mg, or 80 mg lovastatin or a pharmaceutically acceptable salt thereof; 1 mg, 2 mg, or 4 mg pitavastatin or a pharmaceutically acceptable salt thereof; or 20 mg, 40 mg, or 80 mg fluvastatin or a pharmaceutically acceptable salt thereof.

The following disclosure is provided without being limited to any mechanism and solely to explicate what is understood in the art regarding statin-mediated decrease in circulating LDL. LDL-B (apo-lipoprotein B) is a lipid carrier molecule, manufactured in the liver, that is highly atherogenic. LDL-B levels are regulated by liver LDL receptors that bind to circulating LDL particles, resulting in their absorption and ultimate destruction in liver. The greater the density of LDL receptor sites on liver cell (hepatocyte) surfaces, the lower the level of LDL-B cholesterol in circulation. PCSK9 (proprotein convertase subtilisin/kexin type 9) is a glycoprotein expressed by the liver that degrades LDL receptors. When this occurs, LDL-B levels rise in circulation. Statins, by inhibiting HMG CoA reductase, reduce intra-cellular cholesterol production in the hepatocyte, in turn activating SREBP-2 (sterol regulatory element binding protein-2). This pathway then upregulates hepatic LDL receptor sites, increasing liver clearance of circulatory LDL-B. Although statins also upregulate PCSK9 levels by 7%, this increase is more than offset by the upregulation of SREBP-2, resulting in a net decrease in LDL B particles in solution.

Statins also exert an effect on myocytes (skeletal muscle cells) by interfering with intra-cellular cholesterol synthesis, an activity that has no relationship to circulating LDL-B levels. HMB is a metabolite of an essential amino acid (branch chain amino acid leucine), and while it is taken up by myocytes it is not stored in hepatocytes. For this reason, HMB can unexpectedly be used concurrently with statin agents without subverting the hepatic effect of statins in lowering LDL-B cholesterol.

As used herein, the terms “side effect,” “peripheral effect,” and “secondary effect” are interchangeable and refer to effects or symptoms caused by a drug, medication, or pharmaceutical other than its primary, intended effect or indication.

As used herein, the terms “myopathy” and “myopathic” refer to muscle damage, dysfunction, or disease wherein muscle fibers do not function properly for any one of many reasons, resulting in, for example, muscle weakness, muscle cramps, muscle spasms, muscle stiffness, or elevation of creatine kinase (CK or CPK) levels in blood. For example, myositis may be assessed when CK levels rise above a certain amount, such as above a 1 to 10-fold “upper limit of normal” (ULN). In some cases, muscle symptoms might be observed without a concomitant elevation in CK levels. In other cases, CK levels might be elevated without muscle symptoms.

As used herein, the term “rhabdomyolysis” refers to a type of myopathy involving the release of muscle cell products into the bloodstream following muscle cell damage. Some of these muscle cell products, such as myoglobin, are harmful to the kidneys and may lead to kidney damage or kidney failure. Rhabdomyolysis can also result in disseminated intravascular coagulation and/or death. Rhabdomyolysis might be defined, for example, by CK levels above 10,000 IU/liter or above a 10-fold ULN with an elevation in serum creatinine or a need for hydration therapy. Rhabdomyolysis may be statin-induced or non-statin-induced. For example, in some cases rhabdomyolysis is induced by intense exercise. In some embodiments of the methods disclosed herein, HMB administration is used to treat rhabdomyolysis induced by statin administration. In some embodiments, HMB administration is used to treat non-statin-induced rhabdomyolysis.

As used herein, the terms “myalgia” and “myalgic” refer to muscle pain, which may be a symptom of many diseases and disorders, including myopathy.

Myopathy and/or myalgia are the most common side effects associated with the use of statins. Symptoms of statin-induced myopathy include any combination of muscle pain, muscle weakness, or muscle tenderness, such as an aching or cramping sensation in muscles. Tendon pain and nocturnal leg cramping are other possible symptoms. Statin-induced myopathies are typically exacerbated by exercise; thus, athletes are frequently particularly intolerant to statin therapy. The incidence of statin-induced myopathy or myotoxicity is estimated at about 1.5-5% in randomized-control clinical trials.

The pathology of statin-induced myopathy is not fully understood, particularly because multiple pathophysiological mechanisms may contribute to statin myotoxicity. Without being limited to these or any other explanations of how the invention may work or the biochemical or physiological mechanisms or explanations thereof, these mechanisms include statin-induced alterations in muscular membrane composition, isoprenoid and ubiquinone synthesis, mitochondrial function, calcium homeostasis, rate of apoptosis, and atrogin-1 induction.

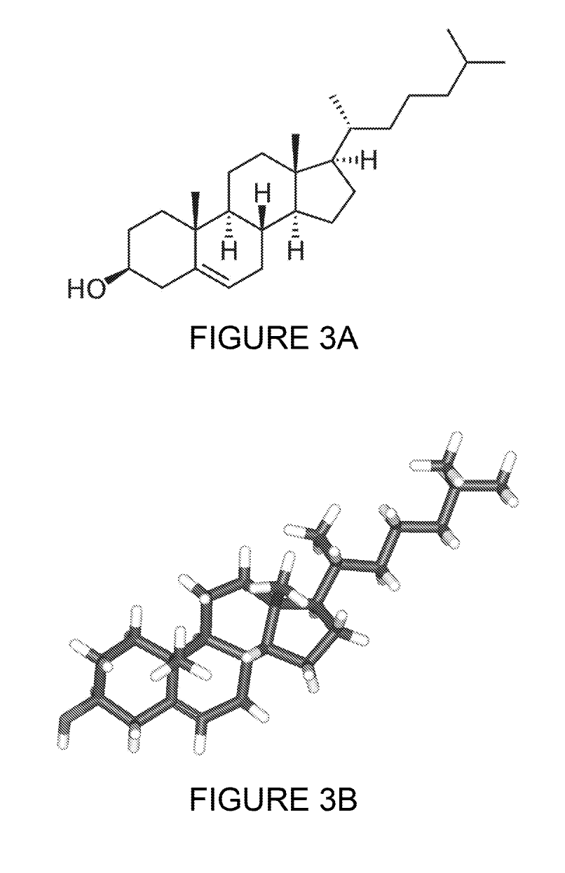

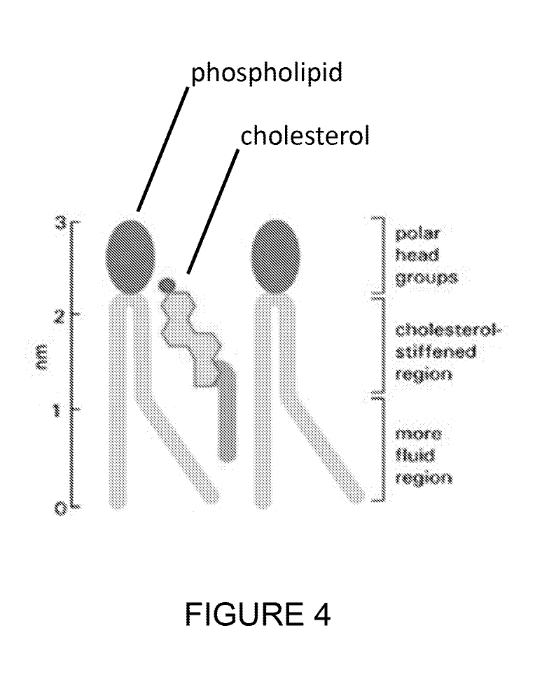

The lipid bilayer of many cell membranes consists not only of phospholipids, but also cholesterol and glycolipids. Eukaryotic plasma membranes contain especially large amounts of cholesterol—up to one cholesterol molecule for every phospholipid molecule. Cholesterol is thus an integral cell membrane component. Cholesterol molecules enhance the permeability-barrier properties of the lipid bilayer and modulate the fluidity of cell membranes, including the membranes of muscle cells. Membrane-bound cholesterol molecules orient themselves in the bilayer with their hydroxyl groups (FIGS. 3A and 3B) toward the polar head groups of the phospholipid molecules (FIG. 4). In this position, cholesterol's rigid, plate-like steroid rings interact with—and partly immobilize—those regions of the phospholipid hydrocarbon chains closest to the polar head groups. By decreasing the mobility of the first few CH₂ groups of the phospholipid hydrocarbon chains, cholesterol makes the lipid bilayer less deformable in this region and thereby decreases the permeability of the bilayer to small water-soluble molecules. Although cholesterol tends to make lipid bilayers less fluid at the high concentrations found in most eukaryotic plasma membranes, it also prevents component hydrocarbon chains from coming together and crystallizing. In this way, it inhibits possible phase transitions. Because statins interfere with cholesterol biosynthesis, they also affect myocyte membrane fluidity. Alterations in membrane fluidity in turn can affect membrane ion channel function, which plays an integral role in membrane excitability. For example, chloride channels in skeletal muscle membranes control resting membrane potential and membrane repolarization. Thus, statin-induced depletion of cholesterol likely disturbs muscle cell function.

According to the isoprenoid synthesis mechanism, statins may cause myopathy by inhibiting synthesis of isoprenoids, for which mevalonate is a precursor. Statin-induced depletion of isoprenoids may in turn disturb cellular respiration, causing myopathy. Under the calcium homeostatis theory of statin-induced myopathy, statin-mediated depletion of isoprenoids leads to decreased inhibition of calcium ion (Ca²⁺) channels in muscle cells, which results in impaired calcium ion homeostasis and impaired myocyte function. Other possible mechanisms of statin-induced myopathy are related to statins' “pleiotropic effects,” which are cholesterol-independent effects of statins. These pleiotropic effects include statin-mediated improvement in endothelial function, stabilization of atherosclerotic plaques, decreases in oxidative stress and inflammation, and inhibition of thrombogenic responses. However, statins can also trigger skeletal muscle apoptosis (i.e. programmed cell death) and, thus, myopathy. Statin-induced myopathy may also be caused through induction, by any statin, of atrogin-1, a human gene that induces muscle pathology directly and is activated by inhibition of the geranasylgeranasyl isoprenoid pathway, part of the cholesterol synthesis cascade obstructed by statins. (See Cao et al., 2009, FASEB J. 23(9):2844-54.)

Statin myopathy appears only in a subset of muscle fibers. In general, the human body consists of a 1:1 ratio of Type 1 (aerobic, slow-twitch) muscle fibers and Type 2 (anaerobic, fast-twitch) muscle fibers. All muscle fibers require cholesterol for cellular repair. Type 2 fibers express LDL receptors, which enable absorption of circulating cholesterol (see Takeda et al., Pathobiology, 2014, 81:94-99). Type 1 fibers, which are used in ordinary activities such as standing and walking, lack LDL receptors and are thus dependent on intracellular cholesterol synthesis, a process inhibited by statin agents. The resulting deficit in cellular cholesterol in Type 1 fibers can lead to statin myopathy.

As used herein, the terms “HMB,” “β-hydroxy β-methylbutyric acid,” “β-hydroxy β-methylbutyrate,” “3-hydroxy-3-methylbutanoic acid,” “3-hydroxy 3-methyl butyrate,” “8-hydroxyisovaleric acid,” and “3-hydroxyisovaleric acid” are interchangeable and refer to the compound of formula (I):

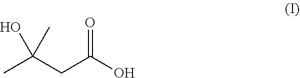

HMB is a metabolite of the amino acid leucine and is synthesized in the human body, where it is converted into the cholesterol precursor HMG-CoA. HMB is used as a dietary supplement by athletes and bodybuilders to enhance performance and training. Daily doses of HMB as a dietary supplement range from about 2 to 5 grams per day, more commonly about 3 grams per day. In terms of dose per body mass, daily doses of HMB as a dietary supplement range from about 17 mg/kg body weight to about 38 mg/kg body weight. Daily HMB dietary supplement dosages can be divided up into, for example, one to four administrations per day.

Toxicologically, the “No Observed Adverse Effect Level” (NOAEL; the highest dose not associated with any toxic signs) for HMB oral ingestion in rats is 3490 mg/kg for male rats and 4160 mg/kg for female rats (see Baxter et al., Chem Toxicol., 2005, 43(12):1731-41). This is an estimated human equivalent of 558 mg/kg and 665 mg/kg for men and women, respectively; assuming a body weight of 150 lbs equates to 38 g (males) and 45 g (females). Human toxicological studies have shown that approximately 6 g HMB daily (78 mg/kg) for one month in untrained young males subject to exercise did not show any toxic effects on serum parameters (half the dose had a spontaneous increase in basophils, considered to be insignificant) and 3 g of HMB daily for up to 8 weeks in both youth and older persons has similarly failed to alter toxicological parameters in serum. This dose has been shown to be safe for one year of administration (see Gallagher et al., Med Sci Sports Exerc., 2000 December; 32(12):2116-19; Nissen et al., J Nutr., 2000 August; 130(8):1937-45). Overall, standard doses of HMB appear to be well-tolerated over long periods of time.

As used herein, the term “leucic acid” refers to the compound of formula (II):

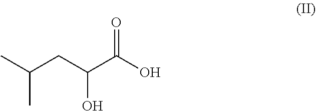

Other, interchangeable, names for leucic acid include L-leucic acid, (S)-leucic acid, 2-hydroxyisocaproic acid (HICA), α-hydroxyisocaproic acid, 2-hydroxy-4-methylpentanoic acid, and 2-hydroxy-4-methylvaleric acid. Leucic acid is a leucine metabolite with reported anti-catabolic and anabolic properties and, as such, is used as a dietary supplement by athletes and bodybuilders to enhance performance and training. Daily doses of leucic acid as a dietary supplement range from about 0.5 to 3 grams per day, more commonly about 1.5 grams per day, with dosages divided up into, for example, one to four administrations per day. See, e.g., Mero et al., 2010, Journal of the International Society of Sports Nutrition 7:1.

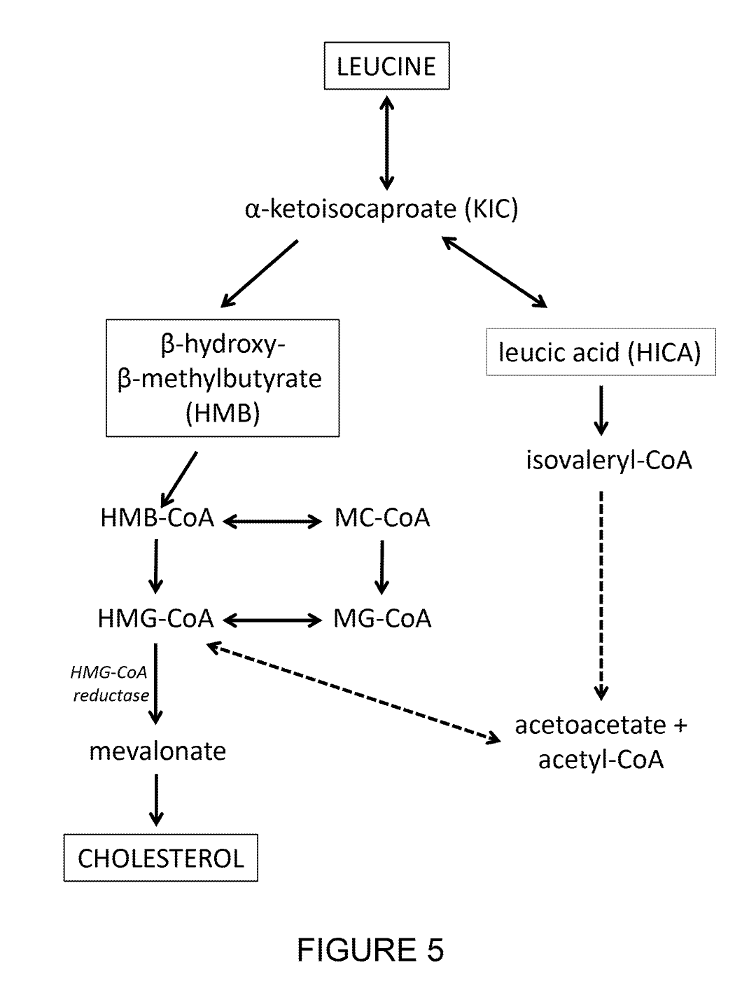

The role of HMB in metabolism of leucine into cholesterol is shown in FIGS. 5 and 6. It takes about 60 grams of leucine to produce 1 gram of HMB; therefore, leucine supplements are ineffective as a source of HMB. Regarding the role of leucic acid in leucine metabolism, leucine is oxidized by an aminotransferase enzyme early in the pathway. The end product of the reaction is keto leucine (α-ketoisocaproate, KIC) as well as leucic acid. The aminotransferase enzyme catalysing this step is capable of oxidizing leucine either to its keto (KIC) or its hydroxyl form (leucic acid) and both reactions are reversible. The reaction between keto and hydroxyl leucine is an equilibrium reaction with an oxidoreduction equilibrium constant (thermodynamic constant) K_{eq}=3.1±0.2×10⁻¹² mol/L and the reaction half time is 230 min towards oxygenation in humans.

As the person of ordinary skill in the art will appreciate, the compounds of the disclosure (e.g., HMB and leucic acid) can be provided as a derivative or prodrug, depending, e.g., on the desired end properties of the compositions and methods. For example, HMB and/or leucic acid may be modified with a suitable prodrug group that metabolizes or otherwise transforms under conditions of use to yield HMB or leucic acid. In one embodiment, the compounds of the disclosure may be modified at the carboxylic acid moiety with a suitable group that can be hydrolyzed. In these embodiments, HMB and/or leucic acid are provided for example as an ester or a lactone. Suitable HMB and/or leucic acid esters include, but are not limited to, methyl ester, ethyl ester, and isopropyl ester. An exemplary, non-limiting HMB lactone includes isovaleryl lactone. HMB and/or leucic acid may also be modified at the hydroxy moiety, for example, with an acetate group. HMB and/or leucic acid derivatives to be used for the compositions and methods of the present disclosure are within the skill of the person skilled in the art using routine trial and experimentation. In some embodiments, HMB derivatives or prodrugs are used in the compositions and methods disclosed herein in order to provide delayed or sustained release of HMB. In other embodiments, leucic acid derivatives or prodrugs are used in the compositions and methods disclosed herein in order to provide delayed or sustained release of leucic acid.

As used herein, the term “hydrate” refers to a compound that is complexed with at least one water molecule. For example, HMB monohydrate refers to a molecule of HMB complexed with one water molecule.

As used herein, the term “alleviate” refers to the amelioration or lessening of the severity of a side effect or symptom or substantially eliminating said side effect or symptom.

As used herein, the term “administer” or “administration” refers to oral (“po”) administration, administration as a suppository, topical contact, intravenous (“iv”), intraperitoneal (“ip”), intramuscular (“im”), intralesional, intranasal or subcutaneous (“sc”) administration, or the implantation of a slow-release device e.g., a mini-osmotic pump, to an individual. Administration can be by any route including parenteral and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, and equivalent methods and modalities know to those of skill in the art.

As used herein, the term “co-administer” refers to administering more than one pharmaceutical agent to a patient. In some embodiments, co-administered pharmaceutical agents are administered together in a single dosage unit. In some embodiments, co-administered pharmaceutical agents are administered separately. In some embodiments, co-administered pharmaceutical agents are administered at the same time. In some embodiments, co-administered pharmaceutical agents are administered at different times.

The term “pharmaceutical formulation” refers to a preparation which is in such form as to permit a biological activity of an active ingredient to be effective, and which contains no additional components which are unacceptably toxic to a subject to which the formulation would be administered.

As used herein, the terms “extended release,” “sustained release,” or “controlled release” refer to compositions that are characterized by having at least one active component having a release profile over an extended period of time, in contrast to “immediate release” pharmaceutical formulations. In some embodiments, the compositions disclosed herein release their active components over a period of about 6 hours to about 72 hours, or about 12 hours to about 48 hours, or about 12 hours to about 36 hours, or about 18 hours to about 30 hours, or about 24 hours. In some embodiments, the active component is released over a time period such that the composition can be administered to a subject once a day, for example, over 24 hours.

In some embodiments, the active ingredients of the compositions and methods disclosed herein are formulated in free acid or free base form. For example, in some embodiments, HMB is formulated as HMB free acid. In some embodiments, HMB free acid is administered orally or sublingually as a gel. In some embodiments, leucic acid is formulated as a free acid. In some other embodiments, leucic acid free acid is administered orally or sublingually as a gel.

In some embodiments, the active ingredients of the compositions and methods disclosed herein are formulated as a pharmaceutically acceptable salt. As used herein, the term “pharmaceutically acceptable salt” refers to salts of the compounds of the present invention derived from the combination of such compounds and a pharmaceutically acceptable organic or inorganic acid (acid addition salts) or a pharmaceutically acceptable organic or inorganic base (base addition salts) which retain the biological effectiveness and properties of the compounds of the present invention and which are not biologically or otherwise undesirable. Examples of pharmaceutically acceptable salts include but not limited to those described in for example: “Handbook of Pharmaceutical Salts, Properties, Selection, and Use”, P. Heinrich Stahl and Camille G. Wermuth (Eds.), Published by VHCA (Switzerland) and Wiley-VCH (FRG), 2002. The compounds of the present invention may be used in either the free base or salt forms, with both forms being considered as being within the scope of the present invention. For example, HMB or leucic acid may be administered as a salt selected from the group consisting of a sodium salt, a potassium salt, a magnesium salt, a chromium salt, and a calcium salt. Other non-toxic salts, such as other alkali metal or alkaline earth metal salts can be used. In some embodiments, HMB may be administered as calcium HMB monohydrate. In some embodiments, leucic acid may be administered as a calcium or sodium salt. Other salts which may act as carriers include succinate, fumarate, and medoximil.

Extended release salts such as succinate may be bound to the active ingredients of the disclosure (e.g., HMB and leucic acid) such that the particular active ingredient is released at a controlled rate. In some embodiments, the particular active ingredient is released at a rate such that the it can be administered once a day. For example, HMB and leucic acid have relatively short half-lives and reach peak levels quickly. Therefore, binding HMB and/or leucic acid to a slow release carrier may have some utility in terms of compliance and efficacy.

HMB and/or leucic acid may be combined with any of the above-mentioned statins to provide combination lipid lowering therapy in patients who are otherwise statin intolerant. Examples would include HMB/atorvastatin, HMB/rosuvastatin, HMB/pravastatin, HMB/simvastatin and HMB/lovastatin, HMB/HICA/atorvastatin, HMB/HICA/rosuvastatin, HMB/HICA/pravastatin, HMB/HICA/simvastatin and HMB/HICA/lovastatin.

In some embodiments of the methods and compositions disclosed herein, and particularly in embodiments where HMB and leucic acid are administered or formulated in a 1:1 ratio, HMB and leucic acid are formulated and/or administered as a conjugate. As used herein, the term “conjugate” refers to two molecular entities joined by one or more bonds, such as covalent bonds, or by another arrangement that provides binding of one molecular entity to the other. For example, HMB and leucic acid may be conjugated together by direct covalent linkage, or by way of a linker, as described below, including a polymer linker, such as an ethylene glycol polymer linker, or a peptide linker comprising one or more amino acid residues. In some embodiments, HMB and leucic acid may be conjugated together by linking one protein to a ligand and linking the second protein to a receptor, e.g., streptavidin and biotin or an antibody and an epitope. In some embodiments, the linker is a cleavable linker formulated such that the conjugated HMB and leucic acid are released from one another once administered to a subject.

For example, conjugates may contain ester linkages that are stable at serum pH but hydrolyse to release the conjugated molecules (such as HMB and leucic acid) from one another when exposed to intracellular pH. Other examples include amino acid linkers designed to be sensitive to cleavage by specific enzymes in the desired target organ. Exemplary linkers are set out in Blattler et al., 1985, Biochem. 24:1517-1524; King et al., 1986, Biochem. 25:5774-5779; and Srinivasachar and Nevill, 1989, Biochem. 28:2501-2509, each of which is incorporated herein by reference in its entirety.

Linkers can contain an alkyl, aryl, polyethylene glycol, polypropylene glycol, hydrazide, and/or amino acid backbone, and further contain an amide, ether, ester, hydrazone, disulphide linkage or any combination thereof. Linkages containing amino acid, ether and amide bound components are generally stable under conditions of physiological pH, normally 7.4 in serum.

In other embodiments, the linker is from 1 to 30 atoms long with carbon chain atoms that may be substituted by heteroatoms that are independently O, N. or S.

In some embodiments, the linker group is hydrophilic to enhance the solubility of the conjugate in body fluids. In some embodiments, the linker contains or is attached to the conjugated molecules by a functional group subject to attack by other lysosomal enzymes. In some embodiments, the conjugated molecules are joined by a linker comprising amino acids or peptides, lipids, or sugar residues.

Representative functional group linkages, of which a linker may have one or more, are amides (—C(O)NR³—), ethers (—O), thioethers- (—S—), carbamates (—OC(O)NR³), thiocarbamates- (—OC(S)NR³), ureas- (—NR³C(O)NR³), thioureas- (—NR³C(S)NR³—), amino groups (—NR³—), carbonyl groups (—C(O)), alkoxy- groups (—O-alkylene-), etc. The linker may be homogenous or heterogeneous in its atom content (e.g., linkers containing only carbon atoms or linkers containing carbon atoms as well as one or more heteroatoms present on the linker. In another embodiment, the linker contains 1 to 25 carbon atoms and 0 to 15 heteroatoms that can be oxygen, NR³, sulfur, —S(O)— and —S(O)₂—, where R³ is hydrogen, alkyl or substituted alkyl. The linker may also be chiral or achiral, linear, branched or cyclic.

Intervening between the functional group linkages or bonds within the linker, the linker may further contain spacer groups including, but not limited to, spacers selected from alkyl, substituted alkyl, aryl, substituted aryl, cycloalkyl, substituted cycloalkyl, heteroaryl, substituted heteroaryl, heterocyclic, substituted heterocyclic, and combinations thereof. The spacer may be homogenous or heterogeneous in its atom content (e.g., spacers containing only carbon atoms or spacers containing carbon atoms as well as one or more heteroatoms present on the spacer. In another embodiment, the spacer contains 1 to 25 carbon atoms and 0 to 15 heteroatoms selected from oxygen, NR³, sulfur, —S(O)— and —S(O)₂—, where R³ is as defined above. The spacer may also be chiral or achiral, linear, branched or cyclic.

Non-limiting examples of spacers are straight or branched alkylene chains, phenylene, biphenylene, etc. rings, all of which are capable of carrying one or more than one functional group capable of forming a linkage with the active compound or research compound. One particular example of a polyfunctional linker-spacer group is lysine, which may link any of the active compounds to two polymer moieties via the two amino groups substituted on a C₄ alkylene chain. Other non-limiting examples include p-aminobenzoic acid and 3,5-diaminobenzoic acid which have 2 and 3 functional groups respectively available for linkage formation. Other such polyfunctional linkage plus spacer groups can be readily envisaged by one of skill in the art.

Reaction chemistries resulting in conjugate linkers are well known in the art. Such reaction chemistries involve the use of complementary functional groups on the linker and the conjugated molecules. It is understood, of course, that if the appropriate substituents are found on the molecules to be conjugated then an optional linker may not be needed as there can be direct linkage of the conjugated compounds.

Table 1 below illustrates numerous complementary reactive groups and the resulting bonds formed by reaction there between. One of ordinary skill in the art can select the appropriate solvents and reaction conditions to effect these linkages.

TABLE 1
Representative Complementary Binding Chemistries

| First Reactive Group | Second Reactive Group | Linkage |
|---|---|---|
| Hydroxyl | Isocyanate | Urethane |
| Amine | Epoxide | β-hydroxyamine |
| sulfonyl halide | Amine | Sulfonamide |
| Carboxyl | Amine | Amide |
| Hydroxyl | alkyl/aryl halide | Ether |
| Aldehyde (under reductive | Amine | Amine |
| amination conditions) | | |

Examples of linkers include, by way of example, the following —O—, —NR³—, —NR³C(O)O—, —OC(O)NR³—, —NR³C(O)—, —C(O)NR³—, —NR³C(O)NR³—, -alkylene-NR³C(O)O—, -alkylene-NR³C(O)NR³—, -alkyleneOC—(O) NR³—, -alkylene-NR³—, -alkylene-O—, -alkylene-NR³C(O)—, -alkyleneC—(O)NR³—, —NR³C(O)O-alkylene-, —NR³C(O)NR³-alkylene-, —OC(O) NR³-alkylene, —NR³-alkylene-, —O-alkylene-, —NR³C(O)-alkylene-, —C(O)NR³-alkylene-, -alkylene-NR³C(O)O-alkylene-, -alkylene-NR³C(O)NR³-alkylene-, -alkyleneOC—(O)NR³-alkylene-, -alkylene-NR³-alkylene, -alkylene-O-alkylene-, -alkylene-NR³C(O)-alkylene-, —C(O)NR³-alkylene-, —NR³C(O)O-alkyleneoxy-, —NR³C(O)NR³-alkyleneoxy-, —OC(O) NR³-alkyleneoxy, —NR³-alkyleneoxy-, —O-alkyleneoxy-, —NR³C(O)-alkyleneoxy-, —C(O)NR³-alkyleneoxy-, -alkyleneoxy-NR³C(O)O-alkyleneoxy- where R³ is as defined above and

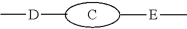

where

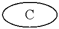

can be aryl, substituted aryl, cycloalkyl, substituted cycloalkyl, heteroaryl, substituted heteroaryl, heterocyclic and substituted heterocyclic, and D and E are independently a
bond, —O—, —CO—, —NR³—, —NR³C(O)O—, —OC(O)NR³—, —NR³C(O)—, —C(O)NR³—, —NR³C(O)NR³—, -alkylene-NR³C(O)O—, -alkylene-NR³C(O)NR³—, -alkyleneOC—(O) NR³—, -alkylene-NR³—, -alkylene-O—, -alkylene-NR³C(O), -alkyleneC-(O)NR³—, —NR³C(O)O-alkylene-, —NR³C(O)NR³-alkylene-, —OC(O)NR³-alkylene-, —NR³-alkylene-, —O-alkylene-, —NR³C(O)-alkylene-, —NR³C(O)O-alkyleneoxy-, —NR³C(O)NR³-alkyleneoxy-, —OC(O) NR³-alkyleneoxy, —NR³-alkyleneoxy-, —O-alkyleneoxy-, —NR³C(O)-alkyleneoxy-, —C(O)NR³-alkyleneoxy-, -alkyleneoxy-NR³C(O)O-alkyleneoxy-, —C(O)NR³-alkylene-, -alkylene-NR³C(O)O-alkylene-, -alkylene-NR³C(O)NR³-alkylene-, -alkyleneOC—(O)NR³-alkylene-, -alkylene-NR³-alkylene-, -alkylene-O-alkylene-, -alkylene-NR³C(O)-alkylene-, or —C(O)NR³-alkylene-, where R³ is as defined above.

Suitable alkylene groups in the above linkers include C₁-C₁₅ alkylene groups, such as C₁-C₆ alkylene groups and C₁-C₃ alkylene groups. Suitable heterocyclic groups include piperazinyl, piperidinyl, homopiperazinyl, homopiperidinyl, pyrrolidinyl, and imidazolidinyl. Suitable alkyleneoxy groups are —(CH₂—CH₂—O)₁₋₁₅—.

In one aspect, the disclosure provides methods for alleviating one or more side effects of statin administration, the method comprising supplementing statin administration with administration of a therapeutically effective amount of β-hydroxy β-methylbutyrate (HMB), in combination with administration of a therapeutically effective amount of leucic acid, wherein one or more side effects of statin administration are alleviated. In another aspect, the disclosure provides methods for alleviating one or more side effects of statin administration comprising co-administering β-hydroxy β-methylbutyrate (HMB) and leucic acid.

In some embodiments, the one or more side effects of statin administration are one or a plurality of myopathic or myalgic side effects, short-term memory loss, abnormal liver function (statin-induced hepatic trans-aminitis), glucose intolerance, hyperglycemia, increased risk for diabetes, or cumulative trauma disorder (also known as chronic overuse syndrome or repetitive overuse syndrome). Thus, side effects of statins as used herein may include both symptomatic and non-symptomatic effects. In some embodiments, the myopathic or myalgic side effects include muscle fatigue, muscle weakness, muscle pain, and/or rhabdomyolysis. In some embodiments, the rhabdomyolysis is acute rhabdomyolysis.

As used herein, the phrases “hepatic trans-aminitis” and “abnormal liver function” refers to liver function characterized by elevated liver functions tests (LFTs), and in particular, elevations in levels of alanine transaminase (ALT, also known as SGPT) and/or aspartate transaminase (AST, also known as SGOT) enzymes. Elevated ALT and AST levels are indicators of liver damage. Other terms for this condition include transaminasemia and transaminitis. LFTs are “elevated” when above the normal ranges, which are about 8-40 U/L for ALT and AST.

As used herein, the phrase “glucose intolerance” refers to a metabolic condition resulting in higher-than-normal levels of blood glucose. Glucose intolerance can include type 1, type 1.5, and type 2 diabetes. Measurement of glycated hemoglobin levels (hemoglobin A1c or HbA1c) in a patient is one way to assess glucose intolerance and/or diabetes. For people without diabetes, the normal range for the hemoglobin A1c test is between 4% and 5.6%. Hemoglobin A1c levels between 5.7% and 6.4% indicate increased risk of diabetes, and levels of 6.5% or higher indicate diabetes. Thus, in some embodiments of the methods disclosed herein, glucose intolerance is characterized by hemoglobin A1c levels at or exceeding about 5.6%, or about 5.7%, or about 6.4%, or about 6.5%.

In another aspect, the disclosure provides methods for treating statin intolerance, the methods comprising supplementing statin administration with administration of a therapeutically effective amount of β-hydroxy β-methylbutyrate (HMB), in combination with a therapeutically effective amount of leucic acid. In another aspect, the disclosure provides methods for treating statin intolerance, the methods comprising co-administering therapeutically effective amounts of β-hydroxy β-methylbutyrate (HMB) and leucic acid. In some embodiments, statin intolerance comprises muscle aches, pains, weakness, or cramps.

As used herein, the term “statin intolerance” refers to effects of statin administration that would lead to discontinuation of the statin therapy if those effects are left unaddressed. The most common presentations of statin intolerance include muscle aches, pains, weakness, or cramps, often called myalgias. In contrast, the term “side effects of statin administration” encompasses any condition resulting from statin therapy including those which can be asymptomatic.

In another aspect, the disclosure provides methods for treating cumulative trauma disorder, the methods comprising administration of a therapeutically effective amount of β-hydroxy β-methylbutyrate (HMB), in combination with a therapeutically effective amount of leucic acid.

In some embodiments of the methods disclosed herein, HMB is administered at a dosage of about 0.5 to about 10 grams/day, or of about 1.0 to about 6.0 grams/day, or of about 2.0 to 4.0 grams/day. In some embodiments, HMB is administered at a dosage of approximately 4 grams/day. In some embodiments, HMB is administered at a dosage of approximately 3 grams/day. In some embodiments, HMB is administered 1 to 5 times per day. In some embodiments, HMB is administered 3 times per day. In some embodiments, HMB is administered 2 times per day. In some embodiments, HMB is administered 1 time per day.

In some embodiments of the methods disclosed herein, leucic acid is administered at a dosage of about 0.5 to about 10 grams/day, or of about 1.0 to about 6.0 grams/day, or of about 1.0 to 4.0 grams/day. In some embodiments, leucic acid is administered at a dosage of approximately 1.5 grams/day. In some embodiments, leucic acid is administered at a dosage of approximately 3.0 grams/day. In some embodiments, leucic acid is administered 1 to 5 times per day. In some embodiments, leucic acid is administered 3 times per day. In some embodiments, leucic acid is administered 2 times per day. In some embodiments, leucic acid is administered 1 time per day.

In some embodiments, HMB and leucic acid are administered as a conjugate.

In some embodiments, HMB is administered as a calcium salt, such as calcium HMB monohydrate. In some embodiments, HMB is administered as HMB free acid. In some embodiments, leucic acid is administered as a sodium salt. In some embodiments, leucic acid is administered as a calcium salt.

In some embodiments of the methods disclosed herein, HMB and/or leucic acid is administered in an extended-release form. In some embodiments, the extended-release form of HMB and/or leucic acid comprises succinate in order to extend release time in the gastrointestinal tract. In some embodiments, extended-release forms of HMB and/or leucic acid are designed or formulated to be administered one to three times per day. In some embodiments, extended-release forms of HMB and/or leucic acid are formulated to be administered once per day.

In some embodiments of the methods disclosed herein, the statin is atorvastatin, cerivastatin, fluvastatin, lovastatin, mevastatin, pitavastatin, pravastatin, rosuvastatin, or simvastatin. In some embodiments, the statin is rosuvastatin.

In another aspect, the disclosure provides methods for treating acute rhabdomyolysis comprising administering a therapeutically effective amount of HMB in combination with a therapeutically effective amount of leucic acid. In some embodiments, the acute rhabdomyolysis is not statin-induced. For example, acute rhabdomyolysis is caused by dehydration, trauma, and/or intense exercise. In some embodiments, HMB is administered at a dosage of from about 3 grams/day to about 15 grams/day and leucic acid is administered at a dosage of from about 1 gram/day to about 15 grams/day. In some embodiments, HMB is administered at a dosage of about 12 grams/day. In some embodiments, HMB is administered at a dosage of 6 grams twice a day. In some embodiments, leucic acid is administered at a dosage of 1.5 grams a day. In some embodiments, leucic acid is administered at a dosage of 3 grams a day. In some embodiments, HMB is HMB free acid. In some embodiments, HMB and leucic acid are administered for at least three days. In some embodiments, HMB and leucic acid are administered as a conjugate.

In another aspect, the disclosure provides pharmaceutical formulations comprising therapeutically effective amounts of a statin, β-hydroxy β-methylbutyrate (HMB), and leucic acid, wherein HMB and leucic acid are present in amounts sufficient to alleviate myopathic or myalgic statin side effects. In some embodiments of the pharmaceutical formulations disclosed herein, the amount of HMB comprises a dosage of HMB of about 0.5 to about 10 grams/day, or of about 1.0 to about 6.0 grams/day, or of about 2.0 to 4.0 grams/day. In some embodiments, the amount of HMB comprises a dosage of approximately 3.0 grams/day. In some embodiments, the amount of HMB comprises a dosage of approximately 4.0 grams/day. In some embodiments, the HMB is HMB monohydrate. In some embodiments, the HMB is HMB calcium salt. In some embodiments of the pharmaceutical formulations disclosed herein, the amount of leucic acid comprises a dosage of leucic acid of about 0.5 to about 10 grams/day, or of about 1.0 to about 6.0 grams/day, or of about 2.0 to 4.0 grams/day. In some embodiments, the amount of leucic acid comprises a dosage of leucic acid of approximately 1.5 grams/day. In some embodiments, the amount of leucic acid comprises a dosage of leucic acid of approximately 3.0 grams/day. In some embodiments, HMB and leucic acid are formulated as a conjugate.

In some embodiments of the pharmaceutical formulations disclosed herein, the statin is atorvastatin, cerivastatin, fluvastatin, lovastatin, mevastatin, pitavastatin, pravastatin, rosuvastatin, or simvastatin. In some embodiments, the statin is rosuvastatin. In some embodiments, the ratio of statin to HMB is approximately 0.001 to 0.1 by weight. In some embodiments, the ratio of statin to HMB is about 0.008, or about 0.01, or about 0.02, or about 0.03, or about 0.04, or about 0.05, or about 0.06, or about 0.07, or about 0.08, or about 0.09, or about 0.1 by weight. In some embodiments, the ratio of statin to HMB is approximately 0.01 by weight. In some embodiments, the amount of HMB is from about 1.0 gram to about 4.0 grams. In some embodiments, the ratio of statin to leucic acid is approximately 0.001 to 0.1 by weight. In some embodiments, the ratio of statin to leucic acid is about 0.008, or about 0.01, or about 0.02, or about 0.03, or about 0.04, or about 0.05, or about 0.06, or about 0.07, or about 0.08, or about 0.09, or about 0.1 by weight. In some embodiments, the ratio of statin to leucic acid is approximately 0.01 by weight. In some embodiments, the amount of leucic acid is from about 1.0 gram to about 4.0 grams. In some embodiments, the amount of leucic acid is about 1.5 grams. In some embodiments, the amount of leucic acid is about 3 grams.

In some embodiments of the pharmaceutical formulations disclosed herein, HMB and/or leucic acid is formulated for extended release. In some embodiments, extended-release forms of HMB and/or leucic acid comprise succinate in order to extend release time in the gastrointestinal tract. In some embodiments, extended-release forms of HMB and/or leucic acid are designed or formulated to be administered one to three times per day. In some embodiments, extended-release forms of HMB and/or leucic acid are formulated to be administered once per day. In some embodiments of the pharmaceutical formulations disclosed herein, HMB and leucic acid are conjugated together.

In another aspect, the disclosure provides uses of HMB in combination with leucic acid to alleviate one or more side effects in a patient administered a statin. In some embodiments, the one or more side effects of statin administration are one or a plurality of myopathic or myalgic side effects, short-term memory loss, abnormal liver function, glucose intolerance, hyperglycemia, increased risk for diabetes, or cumulative trauma disorder. In some embodiments, the myopathic or myalgic side effects include muscle fatigue, muscle weakness, muscle pain, and/or rhabdomyolysis. In some embodiments, the rhabdomyolysis is acute rhabdomyolysis.

In another aspect, the disclosure provides uses of β-hydroxy β-methylbutyrate (HMB) in combination with leucic acid to treat statin intolerance. In some embodiments, statin intolerance comprises muscle aches, pains, weakness, or cramps.

In another aspect, the disclosure provides uses of HMB in combination with leucic acid to treat cumulative trauma disorder.

In some embodiments of the uses disclosed herein, HMB is administered at a dosage of about 0.5 to about 15 grams/day, or of about 1.0 to about 6.0 grams/day, or of about 2.0 to 4.0 grams/day. In some embodiments, HMB is administered at a dosage of approximately 4 grams/day. In some embodiments, HMB is administered at a dosage of approximately 3 grams/day. In other embodiments, HMB is administered at a dosage of about 3.0 to about 15.0 grams/day. In some embodiments, HMB is administered at a dosage of about 12.0 grams/day. In some embodiments, HMB is administered 1 to 5 times per day. In some embodiments, HMB is administered 3 times per day. In some embodiments, HMB is administered 2 times per day. In some embodiments, HMB is administered 1 time per day. In some embodiments, HMB is administered as a calcium salt, such as calcium HMB monohydrate. In some embodiments, HMB is administered as HMB free acid.

In some embodiments of the uses disclosed herein, leucic acid is administered at a dosage of about 0.5 to about 15 grams/day, or of about 1.0 to about 6.0 grams/day, or of about 1.0 to 4.0 grams/day. In some embodiments, leucic acid is administered at a dosage of approximately 1.5 grams/day. In some embodiments, leucic acid is administered at a dosage of approximately 3 grams/day. In some embodiments, leucic acid is administered 1 to 5 times per day. In some embodiments, leucic acid is administered 3 times per day. In some embodiments, leucic acid is administered 2 times per day. In some embodiments, leucic acid is administered 1 time per day. In some embodiments, leucic acid is administered as a calcium salt or as a sodium salt. In some embodiments, leucic acid is administered as a free acid.

In some embodiments of the uses disclosed herein, HMB and/or leucic acid is administered in an extended-release form. In some embodiments, the extended-release form of HMB and/or leucic acid comprises succinate in order to extend release time in the gastrointestinal tract. In some embodiments, extended-release forms of HMB and/or leucic acid are designed or formulated to be administered one to three times per day. In some embodiments, extended-release forms of HMB and/or leucic acid are formulated to be administered once per day. In some embodiments of the uses disclosed herein, HMB and leucic acid are administered as a conjugate.

In some embodiments of the uses disclosed herein, the statin is atorvastatin, cerivastatin, fluvastatin, lovastatin, mevastatin, pitavastatin, pravastatin, rosuvastatin, or simvastatin.

In another aspect, the disclosure provides uses of β-hydroxy β-methylbutyrate (HMB) in combination with leucic acid to treat acute rhabdomyolysis. In some embodiments, HMB is administered at a dosage of approximately 6.0 grams/day to approximately 12.0 grams/day, and leucic acid is administered at a dosage of approximately 1.0 grams/day to approximately 3.0 grams/day. In some embodiments, HMB is administered from 1 to 4 times per day, and wherein leucic acid is administered from 1 to 4 times per day. In some embodiments, HMB is calcium HMB monohydrate. In some embodiments, HMB is HMB free acid. In some embodiments, leucic acid is leucic acid sodium salt. In some embodiments, HMB is conjugated to leucic acid.

### EXAMPLES

The Examples that follow are illustrative of specific embodiments of the invention, and various uses thereof. They are set forth for explanatory purposes only, and are not to be taken as limiting the invention.

### Example 1: Study Evaluating the Effects of HMB and Leucic Acid Combination Therapy on Patients with Statin-Induced Myopathy

The effects of combined administration of HMB and leucic acid was studied in two patients with statin-induced myopathy (SIM). Both patients enrolled were under the care of a board certified cardiologist. In both cases, the patients experienced incomplete resolution of SIM when administered HMB alone. Administration of leucic acid in combination with HMB led to complete resolution of SIM symptoms and reduction of CPK levels to within normal ranges.

Patient A

61 year old male with a history of PCI/coronary stent ramus intermedius; historically statin intolerant lipid disorder with LDL=168, total cholesterol=281, with triglycerides=316 on a hypolipidemic regimen of Welchol, 6 tabs/d (for a total of 1500-3000 mg/d) and Zetia, 10 mg/d; unable to achieve goal (i.e., LDL cholesterol <100 mg % in patients with coronary risk factors and <70 mg % in coronary patients (with previous myocardial infarction (MI), percutaneous coronary intervention (PCI), or coronary bypass surgery) as well as patients with peripheral vascular disease and diabetics) off statins and therefore begun on Vytorin, 10/40 mg daily+3-hydroxy 3-methyl butyrate (HMB) at 2 grams BID. After 4 weeks, LDL dropped to 110 with no recurrence of statin myalgia; after one year on statin+3-hydroxy 2-methyl butyrate, patient remained free of statin myalgia and LDL dropped further to 89. However, 19 months after initiating statin/HMB combination therapy, diffuse myalgia returned. He was continued on his combination therapy but leucic acid, 3 grams/d was added with complete resolution of symptoms within one week.

Patient B

62 year old female with angiographic small vessel disease; type 2 diabetes and type 2a hyperlipidemia; history of statin intolerance with myalgia, weakness and severely elevated CPKs (900-1000 range, normal 200); patient therefore treated with Welchol 6 grams/d and Zetia 10 mg/d with an LDL=185 on this regimen. The patient was subsequently placed on Crestor 20 mg/d+HMB 2 grams BID with a drop in LDL=49 and improved CPK levels (300-400 range). Leg myalgia improved significantly but persisted. Leucic acid at 3 grams/d was then added with complete resolution of symptoms within one week apart from mild discomfort climbing stairs; able to walk 3 miles daily without discomfort; CPK dropped to 200-250 range. Patient evaluated two weeks after beginning leucic acid treatment with no return of previous symptoms and maintained active lifestyle with daily prolonged walking.

### Example 2: In-Patient Treatment of Acute Rhabdomyolysis

HMB and leucic acid co-administration is used to treat acute rhabdomyolysis in an in-patient setting.

Acute rhabdomyolysis is a rare but extreme and potentially life-threatening disorder that can occur in all groups in a setting of dehydration, trauma, and in younger age groups, intense exercise. Statin agents have also been shown to cause acute massive rhabdomyolysis syndrome (a form of statin myopathy) resulting in acute kidney failure and hemodialysis.

Protocol: measure patient's CPK; if value exceeds 10 times the upper limit of normal (200 IU/L, depending on laboratory-specific established normal range), initiate the following:
1. Admit to ICU;
2. Initiate IV 0.9% saline at 165 ml/hour;
3. Initiate HMB free acid (gel form), dose 6 g twice daily for 3 days; a second arm compares HMB alone to HMB 3 g twice daily in combination with leucic acid 1.5 g twice daily with the goal of reducing CPK below 200 IU/L within 3 days.
4. Lab: CPK, BUN, creatinine, glucose and electrolytes every 12 hours for 3 days.

Outcome: HMB and leucic acid co-administration reduces CPK below 200 IU/L within 3 days of initiating HMB/leucic acid treatment.

### Example 3: Treatment of Statin-Induced Short-Term Memory Loss with HMB

HMB administration is used to treat short-term memory loss, an unusual side effect resulting from statin administration. Here, HMB is used in combination with leucic acid to resolve similar short-term memory loss exhibited by patients taking statins.

Protocol: Patient describes short term memory loss after starting statin. Initiate the following steps:
1. Assess memory loss with standardized neuro-psychometric testing as baseline status;
2. Initiate calcium HMB monohydrate, 2 g twice daily; or calcium free acid gel, 1 g three times daily; a second arm compares HMB alone to HMB monohydrate 2 g twice daily in combination with leucic acid 1.5 g twice daily;
3. Continue statin therapy with no dose change;
4. Obtain baseline lab (lipid panel, CPK, liver function/metabolic profile) and repeat in 12 weeks;
5. Reassess memory status in 12 weeks repeating psycho-metric testing and comparing to baseline study.

Outcome: Patients exhibit reversal of some or all short-term memory loss by HMB/Ieucic acid combination treatment within two months. Comparison of pre- and post-therapy neuropsychometric testing will be statistically analyzed (chi-squared, p-value).

### Example 4: Treatment of Statin-Induced Abnormal Liver Function Using HMB

Elevated liver function tests (LFTs) are common in statin users. The presence of elevated transaminases, commonly the transaminases alanine transaminase (ALT or SGPT) and aspartate transaminase (AST or SGOT), are indicators of liver damage. Terms for this condition include hepatic transaminasemia and hepatic transaminitis. Normal ranges for both ALT and AST are 8-40 U/L with mild transaminesemia noted to the upward numerical limit of 250 U/L. Here, HMB is administered to normalize abnormal liver function tests and reverse transaminitis.

Protocol: Patient must exhibit ALT and AST levels in excess of twice the upper limit of normal to enroll. Once enrolled, initiate the following steps:
1. Initiate calcium HMB monohydrate, 2 g twice daily or HMB free acid (gel form), 1 g three times daily; a second arm compares HMB alone to HMB monohydrate 2 g twice daily in combination with leucic acid 1.5 g twice daily;
2. Continue statin agent with no dose change;
3. Repeat liver panel in 3 months and compare to baseline.

Outcome: LFTs return to normal (ALT/SGPT less than 40 U/L, AST/SGOT less than 40 U/L) within 3 months of beginning HMB/leucic acid combination treatment.

### Example 5: Treatment of Statin-Induced Glucose Intolerance Using HMB

Glucose intolerance with hyperglycemia/increased risk for diabetes, probably related to insulin resistance, has been reported in statin users, especially lipophilic statins such as rosuvastatin, less so in hydrophilic statins such as pravastatin. Here, HMB administration is used to treat statin-related glucose intolerance.

Protocol: type 1, 1.5, and 2 diabetics with hemoglobin A1C at or exceeding 6.5% are eligible; also eligible are hyperglycemic/increased risk for diabetes patients not on diabetic treatment but with a hemoglobin A1C at or exceeding 5.7%; patients with critical value fasting blood glucose and hemoglobin A1C levels are excluded. Once enrolled, the following steps are initiated:
1. Continue statin and diabetic medications with dosages unchanged;
2. Initiate calcium HMB monohydrate, 2 g twice daily, or HMB free acid (gel form), 1 g three times daily; a second arm compares HMB alone to HMB monohydrate 2 g twice daily in combination with leucic acid 1.5 g twice daily;
3. Repeat fasting blood glucose and hemoglobin A1C in 3 weeks and 6 weeks;

Outcome: Patients exhibit reduction of hemoglobin A1C to levels below 6.5% in diabetics and 5.7% in hyperglycemics on dietary management (i.e., with increased risk for diabetes) within 6 weeks of beginning HMB/leucic acid combination treatment, along with fasting blood glucose less than 100 mg % for hyperglycemic/non-diabetic management patients and less than 130 mg % for diabetic patients.

### Example 6: Treatment of Statin-Related Cumulative Trauma Disorder Using HMB

Cumulative trauma disorder, also known as chronic overuse syndrome or repetitive overuse syndrome, is characterized by muscle damage due to performing repetitive activities over time. Statin users are particularly vulnerable to cumulative trauma disorder due to impaired ability to heal chronically micro-traumatized muscle. Here, HMB is used to treat cumulative trauma disorder.

Protocol: Patients are enrolled if they qualify with symptoms that include chronic muscle weakness and pain complemented by an occupation or lifestyle lending itself to chronic overuse syndrome (e.g., construction workers, etc.). All patients are currently on statins. Initial documentation of strength levels and pain severity are required. Pain is evaluated using the Verbal Numerical Rating Scale (VNRS). Strength is measured using the Manual Muscle Testing/5-point scale. Additionally, grip strength is measured using a dynamometer. Once enrolled, the following steps are initiated:
1. Obtain baseline lab including lipid panel and CPK-MM levels;
2. Continue statin with dose unchanged;
3. Continue repetitive activity in question at the same level of intensity;
4. Initiate calcium HMB monohydrate, 2 g twice daily, or HMB free acid (gel form, 1 g three times daily; a second arm compares HMB alone to HMB monohydrate 2 g twice daily in combination with leucic acid 1.5 g twice daily;
5. Re-evaluate pain severity and strength levels of patient three weeks and six weeks after enrollment, statistically comparing pain severity and strength levels against baseline (p-value, Chi-squared);
6. Repeat baseline lab six weeks after enrollment.

Outcome: Patients exhibit reduced pain severity and increased strength levels within 6 weeks of beginning HMB/leucic acid combination treatment.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

1. A method for alleviating one or more side effects of statin administration, the method comprising supplementing statin administration with administration of a therapeutically effective amount of β-hydroxy β-methylbutyrate (HMB), in combination with a therapeutically effective amount of leucic acid.

2. The method of claim 1, wherein the one or more side effects of statin administration are myopathic or myalgic side effects, short-term memory loss, elevated alanine transaminase (ALT) or aspartate transaminase (AST) levels, glucose intolerance, hyperglycemia, increased risk for diabetes, or cumulative trauma disorder.

3. The method of claim 1, wherein HMB is administered at a dosage of approximately 2.0 to 4.0 grams/day, and wherein leucic acid is administered at a dosage of approximately 1.0 to 4.0 grams/day.

4. The method of claim 3, wherein HMB is administered at a dosage of approximately 3.0 grams/day.

5. The method of claim 3, wherein HMB is administered at a dosage of approximately 4.0 grams/day.

6. The method of claim 3, wherein leucic acid is administered at a dosage of approximately 1.5 grams/day.

7. The method of claim 3, wherein leucic acid is administered at a dosage of approximately 3.0 grams/day.

8. The method of claim 1, wherein HMB is administered 1 to 5 times per day, and wherein leucic acid is administered 1 to 5 times per day.

9. The method of claim 8, wherein HMB is administered 3 times per day.

10. The method of claim 8, wherein HMB is administered 2 times per day.

11. The method of claim 8, wherein leucic acid is administered 2 times per day.

12. The method of claim 8, wherein leucic acid is administered once per day.

13. The method of claim 1, wherein the statin is atorvastatin, cerivastatin, fluvastatin, lovastatin, mevastatin, pitavastatin, pravastatin, rosuvastatin, or simvastatin.

14. The method of claim 13, wherein the statin is administered at a dosage of:
(a) 10 to 80 mg atorvastatin;
(b) 5 to 40 mg rosuvastatin;
(c) 10 to 80 mg pravastatin;
(d) 5 to 80 mg simvastatin;
(e) 10 to 80 mg lovastatin;
(f) 1 to 4 mg pitavastatin; or
(g) 20 to 80 mg fluvastatin.

15. The method of claim 14, wherein the statin is administered at a dosage of:
(a) 10 mg, 20 mg, or 40 mg atorvastatin;
(b) 5 mg, 10 mg, 20 mg, or 40 mg rosuvastatin;
(c) 10 mg, 20 mg, 40 mg, or 80 mg pravastatin;
(d) 10 mg, 20 mg, or 40 mg simvastatin;
(e) 10 mg, 20 mg, 40 mg, or 80 mg lovastatin;
(f) 1 mg, 2 mg, or 4 mg pitavastatin; or
(g) 20 mg, 40 mg, or 80 mg fluvastatin.

16. The method of claim 1, wherein HMB is administered as calcium HMB monohydrate.

17. The method of claim 1, wherein HMB is administered as HMB free acid.

18. The method of claim 1, wherein leucic acid is administered as leucic acid sodium salt.

19. The method of claim 1, wherein HMB and leucic acid are administered as a conjugate.

20. A method for treating statin intolerance, the method comprising supplementing statin administration with administration of a therapeutically effective amount of β-hydroxy β-methylbutyrate (HMB), in combination with a therapeutically effective amount of leucic acid.

21. The method of claim 20, wherein statin intolerance comprises muscle aches, pains, weakness, or cramps.

22. The method of claim 20, wherein HMB is administered at a dosage of approximately 2.0 to 4.0 grams/day, and wherein leucic acid is administered at a dosage of approximately 1.0 to 4.0 grams/day.

23. The method of claim 22, wherein HMB is administered at a dosage of approximately 3.0 grams/day.

24. The method of claim 22, wherein HMB is administered at a dosage of approximately 4.0 grams/day.

25. The method of claim 22, wherein leucic acid is administered at a dosage of approximately 1.5 grams/day.

26. The method of claim 22, wherein leucic acid is administered at a dosage of approximately 3.0 grams/day.

27. The method of claim 20, wherein HMB is administered 1 to 5 times per day, and wherein leucic acid is administered 1 to 5 times per day.

28. The method of claim 27, wherein HMB is administered 3 times per day.

29. The method of claim 27, wherein HMB is administered 2 times per day.

30. The method of claim 27, wherein leucic acid is administered 2 times per day.

31. The method of claim 27, wherein leucic acid is administered once per day.

32. The method of claim 20, wherein the statin is atorvastatin, cerivastatin, fluvastatin, lovastatin, mevastatin, pitavastatin, pravastatin, rosuvastatin, or simvastatin.

33. The method of claim 32, wherein the statin is administered at a dosage of:
(a) 10 to 80 mg atorvastatin;
(b) 5 to 40 mg rosuvastatin;
(c) 10 to 80 mg pravastatin;
(d) 5 to 80 mg simvastatin;
(e) 10 to 80 mg lovastatin;
(f) 1 to 4 mg pitavastatin; or
(g) 20 to 80 mg fluvastatin.

34. The method of claim 33, wherein the statin is administered at a dosage of:
(a) 10 mg, 20 mg, or 40 mg atorvastatin;
(b) 5 mg, 10 mg, 20 mg, or 40 mg rosuvastatin;
(c) 10 mg, 20 mg, 40 mg, or 80 mg pravastatin;
(d) 10 mg, 20 mg, or 40 mg simvastatin;
(e) 10 mg, 20 mg, 40 mg, or 80 mg lovastatin;
(f) 1 mg, 2 mg, or 4 mg pitavastatin; or
(g) 20 mg, 40 mg, or 80 mg fluvastatin.

35. The method of claim 20, wherein HMB is administered as calcium HMB monohydrate.

36. The method of claim 20, wherein HMB is administered as HMB free acid.

37. The method of claim 20, wherein leucic acid is administered as leucic acid sodium salt.

38. The method of claim 20, wherein HMB and leucic acid are administered as a conjugate.

39. A pharmaceutical formulation comprising therapeutically effective amounts of a statin, β-hydroxy β-methylbutyrate (HMB), and leucic acid, wherein side effects or intolerance induced by administration of only the statin are reduced or alleviated.

40. The pharmaceutical formulation of claim 39, wherein the statin is atorvastatin, cerivastatin, fluvastatin, lovastatin, mevastatin, pitavastatin, pravastatin, rosuvastatin, or simvastatin.

41. The pharmaceutical formulation of claim 40, which comprises:
(a) 10 to 80 mg atorvastatin;
(b) 5 to 40 mg rosuvastatin;
(c) 10 to 80 mg pravastatin;
(d) 5 to 80 mg simvastatin;
(e) 10 to 80 mg lovastatin;
(f) 1 to 4 mg pitavastatin; or
(g) 20 to 80 mg fluvastatin.

42. The pharmaceutical formulation of claim 41, which comprises:
(a) 10 mg, 20 mg, or 40 mg atorvastatin;
(b) 5 mg, 10 mg, 20 mg, or 40 mg rosuvastatin;
(c) 10 mg, 20 mg, 40 mg, or 80 mg pravastatin;
(d) 10 mg, 20 mg, or 40 mg simvastatin;
(e) 10 mg, 20 mg, 40 mg, or 80 mg lovastatin;
(f) 1 mg, 2 mg, or 4 mg pitavastatin; or
(g) 20 mg, 40 mg, or 80 mg fluvastatin.

43. The pharmaceutical formulation of claim 39, wherein the formulation comprises statin and HMB at a statin-to-HMB ratio that is about 0.001 to 0.1 by weight, and wherein the formulation comprises statin and leucic acid at a statin-to-leucic acid ratio that is about 0.001 to 0.1 by weight.

44. The pharmaceutical formulation of claim 39, comprising from about 1.0 gram to about 4.0 grams HMB.

45. The pharmaceutical formulation of claim 39, comprising from about 1.0 gram to about 4.0 grams leucic acid.

46. The pharmaceutical formulation of claim 39, wherein HMB is calcium HMB monohydrate.

47. The pharmaceutical formulation of claim 39, wherein HMB is HMB free acid.

48. The pharmaceutical formulation of claim 39, wherein leucic acid is leucic acid sodium salt.

49. The pharmaceutical formulation of claim 39, wherein HMB is conjugated to leucic acid.

50. A method for treating acute rhabdomyolysis in a patient, the method comprising administering a therapeutically effective amount of β-hydroxy β-methylbutyrate (HMB) and a therapeutically effective amount of leucic acid to the patient.

51. The method of claim 50, wherein HMB is administered at a dosage of about 6 grams/day to about 12 grams/day, and wherein leucic acid is administered at a dosage of about 1 gram/day to about 3 grams/day.

52. The method of claim 51, wherein HMB is administered at a dosage of about 12 grams/day.

53. The method of claim 50, wherein leucic acid is administered at a dosage of about 1.5 grams/day.

54. The method of claim 50, wherein HMB is HMB free acid.

55. The method of claim 50 wherein leucic acid is leucic acid sodium salt.

56. The method of claim 50, wherein HMB and leucic acid are administered for at least three days.

57. The method of claim 50, wherein HMB and leucic acid are administered as a conjugate.

58. A method for treating cumulative trauma disorder in a patient, the method comprising administering a therapeutically effective amount of β-hydroxy β-methylbutyrate (HMB) and a therapeutically effective amount of leucic acid to the patient.

59. The method of claim 58, wherein the cumulative trauma disorder is associated with statin use.

60. The method of claim 58, wherein HMB is administered at a dosage of approximately 2.0 to 4.0 grams/day, and wherein leucic acid is administered at a dosage of approximately 1.0 to 4.0 grams/day.

61. The method of claim 60, wherein HMB is administered at a dosage of approximately 3.0 grams/day.

62. The method of claim 60, wherein HMB is administered at a dosage of approximately 4.0 grams/day.

63. The method of claim 60, wherein leucic acid is administered at a dosage of approximately 1.5 grams/day.

64. The method of claim 60, wherein leucic acid is administered at a dosage of approximately 3.0 grams/day.

65. The method of claim 58, wherein HMB is administered 1 to 5 times per day, and wherein leucic acid is administered 1 to 5 times per day.

66. The method of claim 65, wherein HMB is administered 3 times per day.

67. The method of claim 65, wherein HMB is administered 2 times per day.

68. The method of claim 65, wherein leucic acid is administered 2 times per day.

69. The method of claim 65, wherein leucic acid is administered once per day.

70. The method of claim 58, wherein HMB is HMB free acid or calcium HMB monohydrate.

71. The method of claim 58, wherein leucic acid is leucic acid sodium salt.

72. The method of claim 58, wherein HMB and leucic acid are administered for at least three weeks.

73. The method of claim 58, wherein HMB and leucic acid are administered as a conjugate.

74.-90. (canceled)
